(19) United States
(12) Patent Application Publication
(10) Pub. No.: US 2010/0074797 A1
(43) Pub. Date: Mar. 25, 2010

(54) PROCESS FOR PASTEURIZING CHICKEN LITTER

(75) Inventors: STANLEY L. BROWN, LENOX, IA (US); PHILIP BROWN, BROWNSBURG, IN (US)

Correspondence Address: MCKEE, VOORHEES & SEASE, P.L.C., 801 GRAND AVENUE, SUITE 3200, DES MOINES, IA, 50309-2721, US

(73) Assignee: BROWN BEAR CORPORATION, CORNING, IA (US)

(21) Appl. No.: 12/234,749

(22) Filed: Sep. 22, 2008

(51) Int. Cl. A61L 2/04

(52) U.S. Cl. 422/1

(57) ABSTRACT

This process comprises aerating chicken litter into a first windrow and aerating a second time into a second windrow. Each of the windrows are permitted to achieve a temperature of approximately 130° F. internally. The litter is then spread to completely cover the entire area and a second set of chickens is placed on the litter. The more aeration times that the windrow is made the more the litter is pasteurized and the pathogens and beetles are removed.

### FIELD OF THE INVENTION

This invention relates to a process for pasteurizing chicken litter.

### BACKGROUND OF THE INVENTION

Processes have been used for pasteurizing chicken litter, but these have had insufficient results. One process is to remove the crust of the litter over which chickens have been urinating and defecating upon. The process involves removing the crust and leaving a lower portion of the litter in place. Then the crust is replaced with new litter that overlies the older uncrusted layer. However, this is insufficient in that it does not pasteurize the litter and furthermore it requires additional litter to be spread over the old litter so as to replace the crust. This is an expensive procedure and because the crust is replaced requires additional litter.

Another method of utilizing a process for pasteurizing chicken litter is to place the litter in windrows that are not turned or aerated. This creates a temperature of 130° F. in the center, but does not pasteurize the surface area located at the outside surface of the windrow. It also results in a continual decline in the temperature below 130° F. Furthermore, the windrows are substantially greater in height and also in width than the windrows of the present invention.

Therefore, an object of the present invention is a process for improving the pasteurizing of chicken litter.

The process for pasteurizing chicken litter aerates the litter at least two times and places the litter in a windrow at least two times.

The process of pasteurizing chicken litter provides the further improvement wherein the litter is aerated three times or more times and placed in a windrow at least three or more times before spreading the litter and placing new chickens on the litter.

The process of pasteurizing the chicken litter places the windrow in a separate part of the floor each time that it is aerated.

A further object of the present invention is a process for pasteurizing chicken litter wherein the litter is wet to exceed 30% by weight before the process is begun.

A further object of the process for pasteurizing chicken litter is wherein the windrow is permitted to heat to a minimum of 130° F. at the center. The e outside of the windrow is cooler, sometimes below 130° F.

A further object of the present invention is a process for pasteurizing chicken litter wherein each windrow is aerated two or more times and moved to create a new windrow wherein the new windrow experiences a center temperature of at least 130° F.

A further object of the present invention is an improved process for pasteurizing chicken litter which experiences a reduction in mortality rate of the chickens over that of prior methods.

A further object of the present invention is the provision of a process for pasteurizing chicken litter which experiences an increase of the weight gain per day of the chickens over that of prior methods.

A further object of the present invention is the provision of a process for pasteurizing chicken litter which destroys the bacteria and pathogens by pasteurization over that obtained with prior methods.

A further object of the present invention is a process for pasteurizing chicken litter wherein the litter that can be reused at least six times in a year.

A further object of the present invention is the process for pasteurizing chicken litter which is efficient in operation, durable in use, and reuses litter previously used.

### BRIEF SUMMARY OF THE INVENTION

A process for pasteurizing chicken litter comprises entering an area where a first set of chickens have been defecating and urinating upon a layer of litter. The layer of litter covers the entire area and includes a crust which has an increased bacteria and pathogen level. The process involves aerating a first time litter into first time windrows wherein the litter is piled upon itself in a line. The first windrows are then permitted to set until achieving a temperature of at least 130° F. internally. Then a second aeration is applied to the first windrows and second windrows are formed wherein the litter is piled upon itself in a line. The second windrows are then permitted to set until achieving a temperature of at least 130° F. whereby the litter will have a reduced pathogen level from the increased temperature level. The windrows are then permitted to spread out to completely cover the entire area. A second set of chickens is placed on the litter whereby the second set of chickens will receive a decrease in a death rate over chickens whose litter is not aerated both first and second times and an increase in feed to meat conversion rate in terms of pounds of feed per pounds of bird at the time of slaughter over chickens not aerated with a first and second time.

One feature of the present invention involves aerating a third time the second windrows into third windrows and letting the third windrows set until achieving a temperature of at least 130° F. before the spreading step and the placing step.

The process takes no more than six to twelve days.

According to another feature of the present invention the process may be repeated at least six times with six different sets of chickens.

According to another feature of the present invention the process involves forming the first and second and third windrows having a 2 to 3 foot height and a width of 6 to 9 feet.

According to another feature of the present invention the litter is wet to exceeding 30% by weight.

### BRIEF DESCRIPTION OF THE DRAWINGS

FIG. 1 is a perspective view showing the machine for aerating and putting the litter in a windrow.

FIG. 2 shows a first stage in the process involving partially placing the layer of litter below a windrow.

FIG. 3 shows a removal of the windrow to the right to create a first windrow.

FIG. 4 shows a partial removal of the left and right windrows.

FIG. 5 shows the completed step with three first windrows.

FIG. 6 shows the aeration of the three first windrows into three second windrows.

FIG. 7 shows the aeration of three second windrows into three third windrows.

FIG. 8 shows a cross section of the first, second or third windrows.

FIG. 9 is a graph showing a comparison of temperature over several days.

### DETAILED DESCRIPTION OF THE PREFERRED EMBODIMENT

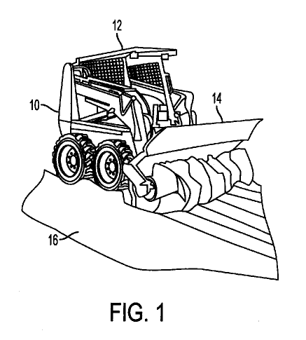

Referring to FIG. 1, a machine 10 is shown having a tractor 12 and a front aerator 14. Front aerator 14 may be mounted at the rear of tractor 12 or at the front of tractor 12. The primary requisite of the aerator is that it be capable of providing an aeration of the entire windrow and also movement of the windrow 16 laterally while at the same time exposing the floor 26 of the shed 27. Several machines 10 are available for aerating and moving the windrow. A preferred machine is made by Brown Bear Corporation, Corning, Iowa.

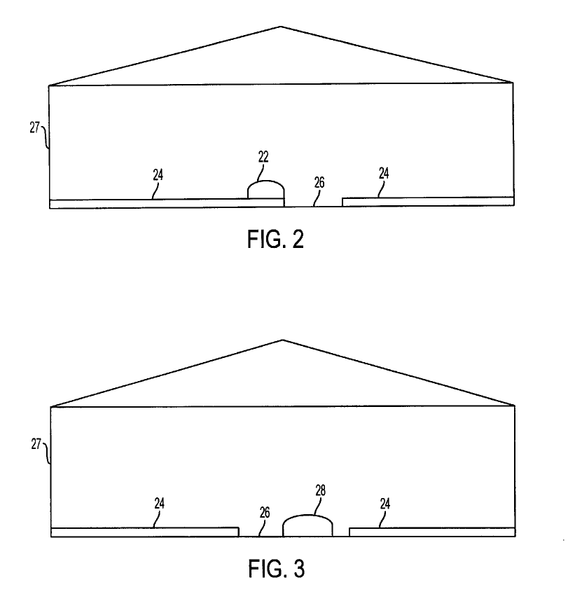

FIGS. 2-7 show a typical shed 27. Referring to FIG. 2 the litter 24 covers the entire floor 26 of shed 27. The litter 24 can be wood chips, sawdust, or other material that is loose, but covers the entire floor 26 of shed 27. A partial windrow 22 is formed by a first pass that removes the litter 24 from the floor 26 and places it upon the litter 24 adjacent the exposed floor 26.

Referring to FIG. 3 a first windrow 28 is formed by again passing the machine 10 along the partial windrow 22 and moving the windrow 28 and the litter 24 below it to the position shown in FIG. 3. This leaves an exposed floor 26.

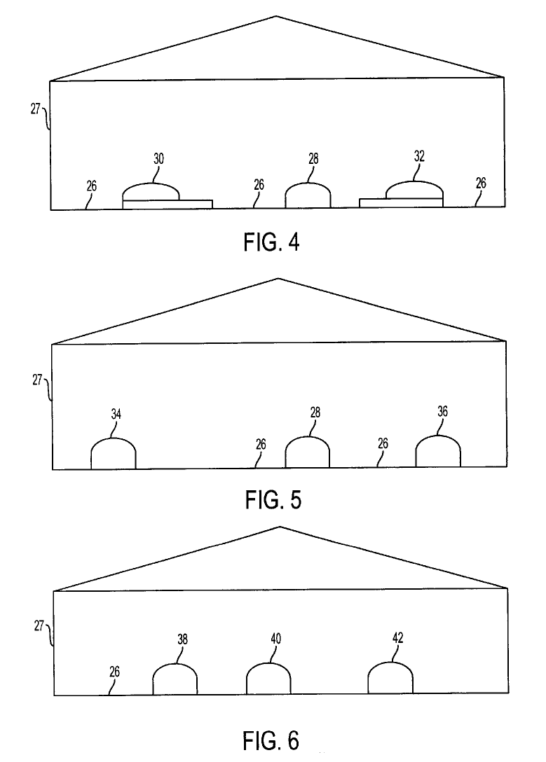

Then a partial windrow 30 and a partial windrow 32 are provided by passing the machine 10 at the outside walls of the floor as shown in FIG. 4. By making passes five and six it is possible to form the first windrow 34, the first windrow 36 and the first windrow 28 as shown in FIG. 5.

FIG. 8 shows leaving of the windrow 28 in the position shown in FIG. 8 for approximately 72 hours. During this time the center portion 50 achieves a temperature of 130° F. or greater. Similarly, the temperature 52 at the outermost regions of the windrow 28 achieves a temperature that is less 130° F. The windrow 28 has a height 1½ to 3½ feet. Similarly the width of the windrow 28 is approximately 6 to 9 feet. This defies earlier judgments that the greater the windrow height and width, the greater temperature would be achieved at hot spot 50. However, it has been found that a height of 1½ to 3½ feet and a width of 6 to 9 feet results in a hot spot 50 that is greater than 130° F.

The process as shown in FIG. 6 shows an additional pass of aeration equipment 14 to create second windrows 38, 40, 42. Essential to the creation of second windrows 38, 40 and 42 is an aeration of the first windrows 28, 34, 36 and also the moving to a portion at floor 26 to create the second windrows 38, 40, 42.

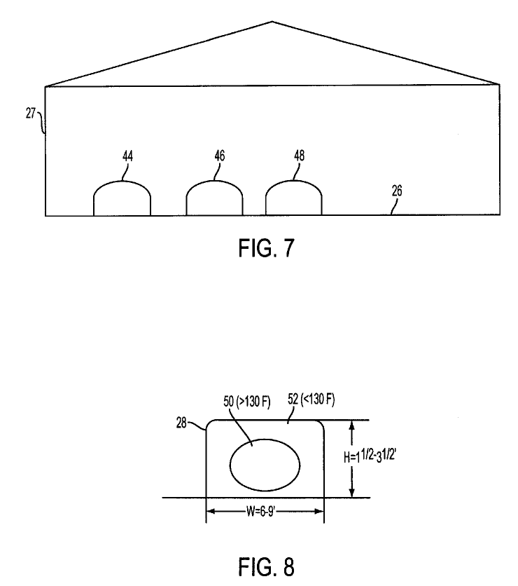

A third turning and aeration of the windrows as shown in FIG. 7 creates a third windrow 44, 46, and 48. Similar to the encounter by machine 10, the windrows 44, 46, 48 should be aerated and also should be removed from the floor 26 to a third position on floor 26.

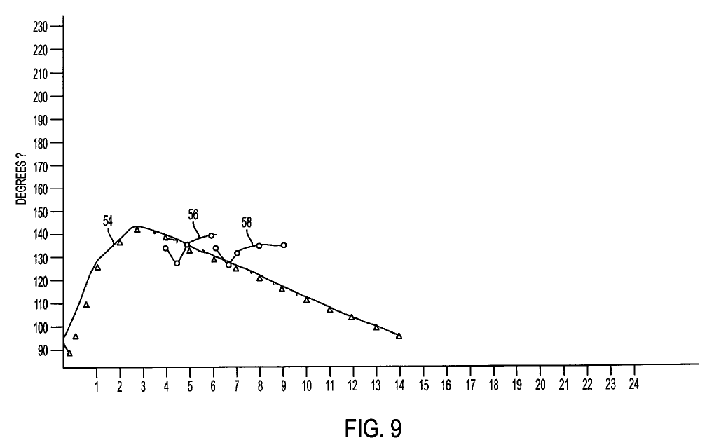

Referring to FIG. 9, a graph 54, a graph 56, and a graph 58 are shown. Graph 54 shows a single windrowing wherein the windrow is not turned. Graphs 56 and 58 show turning the windrow a second and a third time. There can be additional turnings of the windrows, and each turning shows additional pasteurization. However, a second or a third time are preferred.

As can be shown in FIG. 9, the graph 54 quickly heats to in excess of 130° F. in approximately one day. Graph 54 reaches a height of approximately 150° F. and then begins its descent to below 130° F. at approximately day 7. The graph 54 continues its descent below 130° F.

This can be shown in FIG. 8 wherein the hot spot 50 represents the temperature which exceeds 130° F. However, the outside temperature 52 is less than 130° F. and requires additional turning of graphs 56, 58 as shown in FIG. 9. Graph 56 shows a reduction in the temperature of hot spot 50, but quickly shows it increasing to a temperature exceeding 140° F. At the sixth day the material is turned again into a third windrow, and while initially there is a drop in the temperature between the sixth and seventh day, there is an increase thereafter. As can be seen in graphs 56, 58, the temperature of hot spot 50 continues to stay above 130° F. whereas the graph 54 shows a continuing decline. Thus it is possible to turn the graph 54 between the fourth and sixth day as shown in graph 56, and then turn the graph between six and nine days to create a third windrow. While graph 56 is sufficient to pasteurize all of the pathogens, additional windrowing of the third windrow 44, 46, 48 results in increased reduction of pathogens.

Among the factors that affect windrowing, is the bedding material. This is a source of carbon to compost high nitrogen poultry litter. It involves rice hulls, sawdust blend, shavings, or other materials. Rice hulls are the best because they are available in carbon and are good particle size. Shavings suffice because they are high in carbon, but they are sometimes not available, and some have large particle size. It may be possible to combine the rice hulls and the sawdust to compost the material. Other materials may be used.

Another factor is the moisture in the litter. Preferably the moisture in the litter should exceed 30% by weight. 35 to 60% is ideal, but 65% or exceeding that is the least desirable.

How the deep the litter is is also important. Preferably there is less than 4 inches and they should be placed in two or three windrows in 36 to 43 foot wide houses. Four to six inches is ideal. Exceeding six inches should have some material taken to the litter shed for further composting or for composting mortalities.

How long since the house was totally cleaned out to the floor is also important. After 4-6 sets of chickens the house should be cleaned out to the ground and then start composting the next flock.

The time the grower has between flocks 21 to 28 days is ideal. More percentage of disease bacteria and pathogen is killed using this process and the nutrients are stabilized. This is best for beetle control.

20-11 days also will work. There is potential for ammonia and may need some litter amendment. Some beetle control but not as good as 21-28 days.

11-4 days is still good. Possible high ammonia and may need more litter amendment. Good beetle control.

Windrows should be turned every three to five days for 21-28 days and then as needed to dry litter below the 35% moisture area. Beatles, mites, and disease bacteria and pathogens should be eliminated by this process.

Before windrowing poultry houses, all water and feed lines need to be raised as high as they can go. Old cables, cords, box heaters, sorting tubes, trash and curtains should be cleaned out of the litter.

The house should be washed or blown down so that bacteria and pathogens and bugs in the dust and cobwebs also can get composted so as to not reinfect the house.

The advantages of turning windrows are that the turning assures aerates the litter and gets more of the windrow in the core heat zone. It also provides better pathogen reduction, and moves the windrow so that the floor will dry out under the windrow. Flash off of steam or moisture and the ammonia gas is also achieved. The process breaks up chunks of the cake, and blends dry with wet materials. An advantage involves the trapping of beetles and beetle larvae in the core heat zone.

In the drawings and specification there has been set forth a preferred embodiment of the invention, and although specific terms are employed, these are used in a generic and descriptive sense only and not for purposes of limitation. Changes in the form and the proportion of parts as well as in the substitution of equivalents are contemplated as circumstance may suggest or render expedient without departing from the spirit or scope of the invention as further defined in the following claims.

What is claimed is:

1. A process for pasteurizing chicken litter comprising:
entering an area where a first set of chickens have been defecating and urinating upon a layer of litter, the layer of litter covering the entire area and having an increased bacteria and pathogen level;
aerating a first time the litter into a first windrow wherein the litter is piled upon itself in a line;
letting the first windrow set until achieving a temperature of at least 130° F. internally;
aerating a second time the first windrow into a second windrow wherein the litter is piled upon itself in a line;
letting the second windrow set until achieving a temperature of at least 130° F., whereby the litter will have a reduced pathogen level from the increased pathogen level;
spreading the second windrow out to completely cover the entire area;
placing a second set of chickens on the litter, whereby the second set of chickens will receive at least a decrease in a death rate compared to chickens on the litter not aerated both a first and second time, and an increase in a conversion in feed production rate in pounds of feed per pounds of bird at time of slaughter over the chickens on the litter not aerated both a first and a second time.

2. The process according to claim 1 and further comprising aerating a third time the second windrow into a third windrow and letting the third windrow set until achieving a temperature of at least 130° F. before the spreading step and the placing step.

3. The process according to claim 2 wherein all of the process takes no more than 6-12 days.

4. The process according to claim 1 wherein all of the process takes no more than 6-12 days.

5. The process according to claim 1 and further comprising repeating the entire process at least 6 times with six different sets of chickens.

6. The process according to claim 1 and further comprising forming first and second elongated windrows having 1½ to 3½ foot height and 6-9 feet width.

7. The process according to claim 2 and further comprising forming the first, second, and third windrow having a 1½ to 3½ foot height and 6-9 feet width.

8. The process according to claim 1 and further comprising wetting the litter to exceed 30% by weight.

9. A process for pasteurizing chicken litter comprising:
entering an area where a first set of chickens have been on a litter covering the area;
making the litter into a first windrow wherein the litter is piled upon itself in a line and has a height of from 2 to 3 feet and a width of from 6 to nine feet;
letting the first windrow set until achieving a temperature of at least 130° F. internally;
making the litter into a second windrow wherein the litter is piled upon itself in a line, is moved to another position on the floor, and has a height of from 2 to 3 feet and a width of from 6 to nine feet;
letting the second windrow set until achieving an internal temperature of at least 130° F., whereby the litter will have a reduced pathogen level from the increased pathogen level;
spreading the second windrow out to completely cover the area;
placing a second set of chickens on the litter, whereby the second set of chickens will receive at least a decrease in a death rate of chickens on the litter not aerated both a first and second time, and an increase in a conversion in feed production rate of feed per pounds of the second chickens at time of slaughter over chickens on the litter not aerated both a first and a second time.

10. The method of claim 9 and further comprising aerating a third time the second windrow into a third windrow and letting the third windrow set until achieving a temperature of at least 130° F. before the spreading step and the placing step.

11. A process for pasteurizing chicken litter comprising:
entering a building where a first set of chickens have been on a litter that covers a floor of the building;
making the litter into at least one first windrow wherein the litter is piled upon itself in a line;
letting the first windrow set until achieving a temperature of at least 130° F. internally;
making the litter into at least one second windrow wherein the litter is piled upon itself in a line;
moving the second windrow to a second part of the floor that is not occupied by the first windrow;
letting the second windrow set until achieving an internal temperature of at least 130° F.;
moving the second windrow to at least one third windrow and moving the third window to a part of the floor that is not occupied by the second windrow;
letting the third windrow set until achieving and internal temperature of at least 130° F., whereby the litter will have a reduced pathogen level from the increased pathogen level;
spreading the third windrow out to completely cover the area;
placing a second set of chickens on the litter, whereby the second set of chickens will receive at least a decrease in a death rate of chickens on the litter not aerated both a first and second time, and an increase in a conversion in feed production rate of feed per pounds of the second chickens at time of slaughter over chickens on the litter not aerated both a first and a second time.

12. The process according to claim 11 and further comprising repeating the entire process at least 6 times with six different sets of chickens.

13. The process according to claim 11 and further comprising wetting the litter to between 15% to 30% by weight.
